(19) United States
(12) Patent Application Publication
(10) Pub. No.: US 2022/0325003 A1
(43) Pub. Date: Oct. 13, 2022

(54) PARTICLES OF DRIED MICROFIBRILLATED CELLULOSE AND THE USE THEREOF

(71) Applicant: Stora Enso OYJ, Helsinki (FI)

(72) Inventors: Gisela Cunha, Nacka (SE); Kaj Backfolk, Lappeenranta (FI)

(73) Assignee: Stora Enso OYJ, Helsinki (FI)

(21) Appl. No.: 17/595,989

(22) PCT Filed: Jun. 18, 2020
(86) PCT No.: PCT/IB2020/055675

§ 371 Date: Dec. 1, 2021

(30) Foreign Application Priority Data
Jun. 20, 2019 (SE) 1950771-4

(51) Int. Cl. C08B 1/00; A61K 8/73

(52) U.S. Cl. CPC C08B 1/003; A61K 8/731; A61K 2800/28

(57) ABSTRACT

Hornificated microfibri Mated cellulose (MFC) particles are provided, which are useful as an abrasive material in personal care products. Personal care products comprising such particles are also provided.

### TECHNICAL FIELD

This technology provides hornificated particles of microfibrillated cellulose (MFC) that can function as an abrasive material (e.g. an exfoliating agent) in personal care products, representing sustainable alternatives to plastic microbeads.

### BACKGROUND

Plastic microbeads have been used for several years as exfoliation agents in personal care products, such as cosmetics, soaps, facial scrubs and toothpastes. However, such microbeads are usually small (less than 1 mm), and when washed down the drain can pass unfiltered through sewage treatment plants, making their way into rivers and water canals, which culminates in severe microplastic water pollution and endangerment of marine ecosystems worldwide. The plastics from which the microbeads are formed are typically from fossil-fuel based sources, e.g. polyethylene, and are typically not biodegradable.

As a consequence, several countries have banned the use of plastic microbeads in personal care products, and manufacturers are now seeking more environmentally friendly alternatives.

Known alternatives to plastic microbeads used as abrasive components of personal care products include organic materials such as ground fruit/nut kernels, cellulose grains or wax beads (jojoba beads, synthetic wax, carnauba beads, candelilla beads), and inorganic materials such as silica or pumice stone.

However, such abrasive materials often have drawbacks. For instance, inorganic materials are typically difficult to grind, are typically highly abrasive and are usually dense, making them difficult to maintain in suspension. Many fruit or nut kernels have a dark colour, which can impact the colour of the personal care product. Other abrasive materials are difficult to obtain in bulk or can present issues of toxicity or chemical intolerance when applied to the human body.

Various uses of cellulose fibres in skincare or cosmetic applications are provided in WO 2018/030392, WO2002/022172, US2013330417, EP3081209 and JP2006240994A. FR3017291A1 discloses cellulose exfoliant particles that disintegrate after application to human skin or scalp.

There is a need, however, for a new class of abrasive materials for use in personal care products which overcome some or all of the problems with known abrasive materials. First and foremost, the abrasive materials must be biodegradable. Importantly, the abrasive materials should be skin-friendly (i.e. non-toxic). Ideally, the abrasive materials should be a by-product of waste material, a side stream, fractionated or selected stream from industrial processes. The abrasive materials should be easy to grind and allow a range of abrasiveness (provided by both particle size and shape). The abrasive materials should be stable during the lifetime of the product (both physically stable and chemically stable) and should provide stable personal care products. Properties such as flavour, odour and colour should be as neutral as possible. Suitably, the abrasive materials should also have some porosity, which promotes uptake of chemical components.

### SUMMARY

The present technology relates to hornificated particles comprising at least 50 wt % of microfibrillated cellulose (MFC), wherein said particles having a dryness level of 61% or more and a water absorption capacity of less than 10 g water/g material.

A personal care composition is also provided, comprising the hornificated MFC particles described herein. A method for producing hornificated particles comprising or consisting of microfibrillated cellulose (MFC), is also provided, said method comprising the steps of:
drying a composition comprising or consisting of at least 50 wt % of microfibrillated cellulose (MFC), so as to provide hornificated particles having a dryness level of 61% or more,
optionally milling and/or sieving said hornificated particles.

Further provided is the use of hornificated particles comprising or consisting of microfibrillated cellulose (MFC), as described herein, as an abrasive material in a personal care composition.

Further aspects of the present technology are provided in the following text, figures and the dependent claims.

### LEGENDS

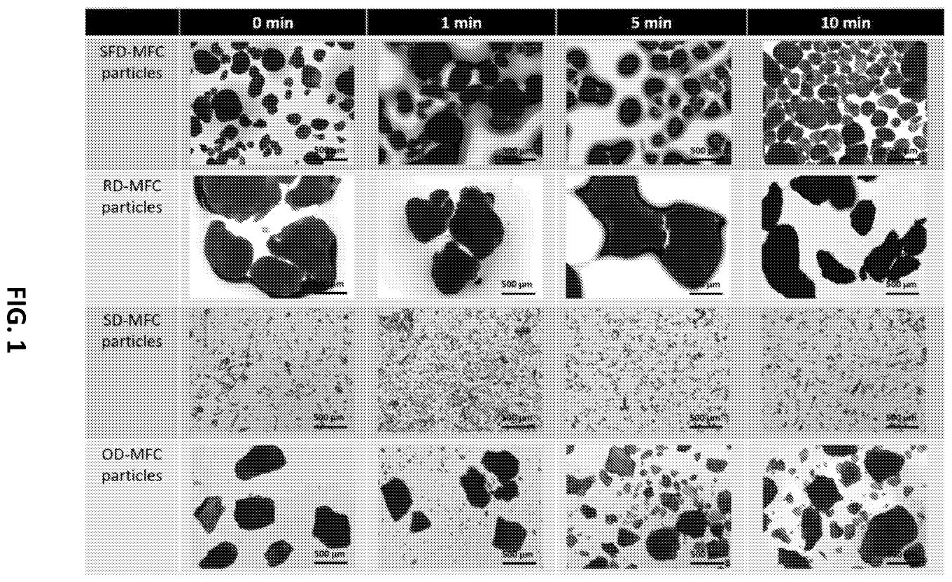

FIG. 1 shows the results of redispersion tests for various hornificated MFC particles

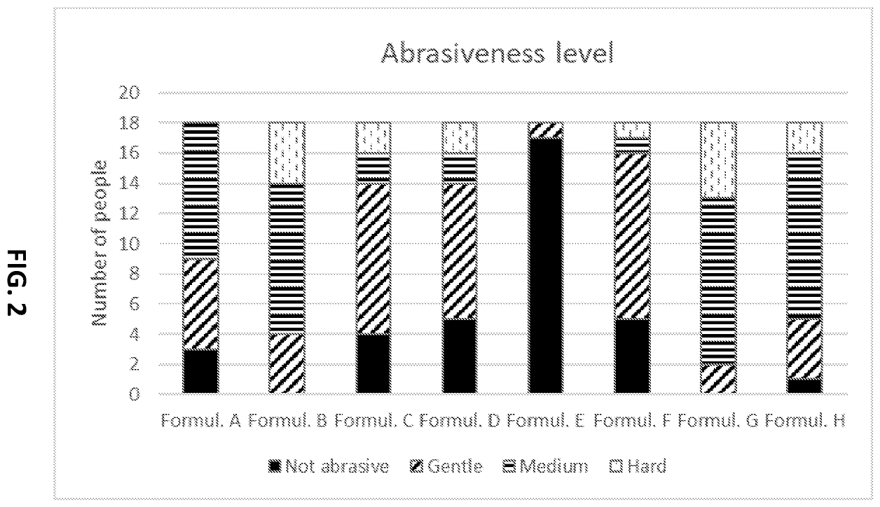

FIG. 2 show the results from a panel evaluation of the abrasiveness level of formulations containing MFC particles

### DETAILED DISCLOSURE

It is known in the forestry and papermaking industries that harsh, extensive drying of cellulose fibers or fibrils will cause them to hornificate. Hornification involves adhesion of fiber surfaces to each other as a result of drying, leading to lower porosity and poor solvent accessibility. Cellulose fibrils aggregate strongly, and thus become virtually impossible to completely separate them again (also known as co-crystallization). Hornificated particles are thus coalesced fiber aggregates or microfibril or elementary fibrils. They are obtained by controlled drying of aggregation of nanofibrils into beads or larger particles. Post-treatment (e.g. post-curing) can increase the extent of hornification. Hornificated particles is not easily redispersable in a solution and this is the purpose of the particles according to the invention, i.e. they do not redisperse in a solution, e.g. an aqueous solution.

Hornification is most often an undesirable feature. At the end of a fibre drying process the temperature of the material starts to increase, because there is no more water to evaporate. Consequently, over drying and hornification can thus occur. Hornification of fibers in e.g. a paper sheet generally leads to the strength properties of the paper sheet being reduced.

Research often focusses on how to dry cellulose fibers or fibrils gently, without causing hornification, so the fibrous material can be re-dispersed again after drying. Known methods for preventing hornification include surface-treatment of fibres prior to drying, or incorporation of antiflocculation agents in the drying process/prior to drying. The present technology is based on the phenomenon of poor re-dispersion of hornificated fibres to produce abrasive materials, which can be used as exfoliant agents.

In one aspect, therefore, hornificated particles comprising or consisting of microfibrillated cellulose (MFC), preferably native microfibrillated cellulose (MFC), are provided. The particles comprise at least 50 wt % MFC, preferably at least 70 wt % MFC, or 100 wt % MFC. Typically, the particles solely comprise cellulosic fibres. The particles may therefore—apart from the MFC—additionally comprise cellulosic fibres such as e.g. pulp fibers. The particles may also comprise other kind of microfibrillated cellulose than native MFC, such as chemically modified MFC.

The MFC particles can also contain fines or highly refined pulp; such as pulp having a Schopper Riegler value between 30 and 90. The particles might also contain microcrystalline cellulose.

Microfibrillated cellulose (MFC) comprises partly or totally fibrillated cellulose or lignocellulose fibers. The liberated fibrils typically have an average diameter less than 100 nm, whereas the actual fibril diameter or particle size distribution and/or aspect ratio (length/width) depends on the source and the manufacturing methods. The smallest fibril is called elementary fibril and has an average diameter of approximately 2-4 nm (see e.g. Chinga-Carrasco, G., Nanoscale research letters 2011, 6:417), while it is common that the aggregated form of the elementary fibrils, also defined as microfibril, is the main product that is obtained when making MFC e.g. by using an extended refining process or pressure-drop disintegration process (see Fengel, D., Tappi J., March 1970, Vol 53, No. 3.). Depending on the source and the manufacturing process, the length of the fibrils can vary from around 1 to more than 10 micrometers.

There are different acronyms for MFC such as cellulose microfibrils, fibrillated cellulose, nanofibrillated cellulose, fibril aggregates, nanoscale cellulose fibrils, cellulose nanofibers, cellulose nanofibrils, cellulose microfibers, cellulose fibrils, cellulose nanofilaments, microfibrillar cellulose, microfibril aggregrates and cellulose microfibril aggregates. MFC can also be characterized by various physical or physical-chemical properties such as large surface area or its ability to form a gel-like material at low solids (1-5 wt %) when dispersed in water.

The microfibrillar cellulose may contain some hemicelluloses; the amount is dependent on the plant source. Mechanical disintegration of the fibers is carried out with suitable equipment such as a refiner, grinder, homogenizer, colloider, friction grinder, single- or twin-screw extruder, fluidizer such as microfluidizer, macrofluidizer or fluidizer-type homogenizer.

MFC can be produced from wood cellulose fibers, both from hardwood or softwood fibers. It can also be made from microbial sources, agricultural fibers such as wheat straw pulp, bamboo, bagasse, or other non-wood fiber sources. It is preferably made from pulp including pulp from virgin fiber, e.g. mechanical, chemical and/or thermomechanical pulps. It can also be made from broke or recycled paper. The term MFC includes parenchymal MFC and BNC (bacterial nanocellulose). MFC can also be obtained from vegetable fibers, e.g. sugar beet or potato based MFC.

The MFC used in the present technology is preferably “native”, i.e. it has not been chemically modified prior to the hornification process.

Hemicellulose content of the MFC fibers is typically 1-25%, while it is not limited by these values. MFC crystallinity is preferably 35-85% and more pref. 45-75%. Various grades of MFC, including those already commercially available and currently on the market, can be used as starting material for the preparation of the dry MFC particles of the invention.

Hornification is a result of harsh drying conditions. The hornificated particles therefore have a dryness level of 61% or more. Suitably, hornificated particles have a dryness level of 70% or more, and preferably 80% or more, more preferably 90% or more, most preferably 95% or more. Dryness level can be determined by an oven-drying method, e.g. ISO/CD 638-1 “Paper, board and pulps—Determination of dry matter content”.

The extent of hornification may also be characterised by the water absorption capacity of the MFC particles, defined as g water uptake/g material. Water absorption capacity can be determined by EDANA method NWSP 240.0.R2 in which saline solution is replaced by deionized water. Hornificated particles have a water absorption capacity of less than 10 g water/g material; preferably less than 5 g water/g material; more preferably 1-5 g water/g material.

The degree of abrasiveness may be related to the particle size and shape, with larger and more angular particles generally providing a more abrasive feel. Depending on the end product, smaller or bigger particles are desirable. Therefore, hornificated particles according to the present technology, may have an average dry particle size (D90) of 1-2000 μm, preferably 50-1000 μm, and more preferably 150-750 μm. Particle size may be measured by laser diffraction (as per ISO/DIS 13320) or by SEM imaging combined with particle analysis (as per ISO 13322-1:2014), preferably laser diffraction.

The cellulosic nature of the MFC particles provides a certain level of porosity, e.g. a porosity in the range 0-25%, preferably in the range 5-15%. Porosity can be determined by mercury porosimetry and gas adsorption as per ISO 15901-1:2016. This is useful when the particles are used in personal care compositions, as it improves uptake of other components of the composition, and the particle's overall compatibility and dispersibility within the composition. Encapsulation or incorporation of additives is not generally possible when grinding cellulose to round-shaped beads.

The degree of abrasiveness may be related to the particle hardness. Hornificated particles suitably have a hardness in the range 60-80 Shore D. The abrasiveness of exfoliants depends on their size and shape. The smallest particles are usually used in facial scrubs, whereas the medium-sized particles are used in body scrubs and finally the biggest particles in foot scrubs.

Another parameter of interest is the surface area. Suitably, the surface area should be >1 m²/g, suitably between 1-1000 m²/g. This is to be compared with larger microcrystalline cellulose particles (180-230 μm) which have a surface area 0.25-0.5 m²/g. Calculating the specific surface area of solids can be carried out by the BET method (e.g. using ISO 9277).

The degree of hornification may also be characterised by the amount of fibrils released by the particles upon wetting. For the hornificated particles according to the present invention the percentage of loose fibrils upon wetting is <5%. Dispersibility in aqueous media can be determined via e.g. Canadian standard CAN/CSA-Z5100-17 5.3.10.

As mentioned above, antiflocculating agents are typically included when drying fibres, so that hornification is reduced or prevented entirely. In the present technology, however, hornification is desired, so the MFC particles preferably do not comprise an antiflocculating agent or debonder. On the other hand, such antiflocculating agents or debonders might be added in a post-treatment step, since it is desired that the MFC particles are stabilized or to adjust rheological behavior, fluidity etc. Additionally, a salt or charge control agent may be added post-drying to reduce the possibility of electrostatic explosion.

MFC particles according to the present invention are bio-based, biodegradable and possess light color (white to yellowish). They are also tuneable in terms of their size, shape and hardness/abrasiveness, depending on the drying technology used, temperature of drying (drying rate), MFC grade and initial solids content. MFC particles are also non-toxic and can be readily produced (upcycled) from by-products of the papermaking and forestry industries. MFC can also be an accept or reject fraction from the fiber fractionation or screening process.

The MFC particles may comprise one or more additives, which are incorporated within the hornificated particles. Suitable additives include surfactants, solvents, oils, proteins, vitamins, pharmaceuticals, pigments etc.

In particular, the MFC particles may comprise one or more pigments, which could be incorporated into the particles prior to drying, during the drying process or post-drying. As the MFC particles themselves do not have a strong colour, the colour of any pigment will be “true”, i.e. not significantly affected by any colour of the particles themselves.

In another aspect, a personal care composition comprising the hornificated MFC particles is provided, where the particles are as defined herein. Examples of personal care products are toothpastes, face scrubs, body scrubs, foot scrubs, and bath and shower products

The personal care composition may be in the form of a liquid, wherein the hornificated MFC particles are dispersed in said liquid. The definition “liquid” includes semi-liquids such as gels or creams. The personal care composition may also be in the form of a solid, e.g. a bar soap, wherein the hornificated MFC particles are dispersed throughout said solid. Typical additives in personal care products include surfactants, rheology modifiers, humectants, pigments etc.

In addition to the hornificated MFC particles, the personal care composition may further comprise non-MFC particles, i.e. particles which do not comprise MFC. This allows the exfoliating properties of the personal care composition to be tailored as required with known exfoliants.

A method is also provided for producing hornificated particles comprising or consisting of microfibrillated cellulose (MFC), said method comprising the steps of:
drying a composition comprising or consisting of at least 50 wt % of microfibrillated cellulose (MFC), so as to provide hornificated particles having a dryness level of 61% or more,
optionally milling and/or sieving said hornificated particles.

The composition comprising or consisting of microfibrillated cellulose (MFC) may be pure MFC or may additionally comprise cellulosic fibres such as e.g. pulp fibers. The composition may also comprise chemically modified MFC or pulp fibers. The composition may also include one or more additives, which—if added prior to the drying step—may become incorporated within the hornificated particles upon drying. Suitable additives include surfactants, rheology modifiers, humectants, pigments, proteins, vitamins, pharmaceuticals etc.

The composition comprises at least 50 wt % MFC, preferably at least 70 wt % MFC, or 100 wt % MFC.

The method provides hornificated particles with a particle size—or which are milled to a particle size—of 1-2000 μm, preferably 50-1000 μm, and more preferably 150-750 μm.

The drying step may comprise spray drying, ring drying, flash drying, TurboRotor mill drying, oven drying, or a combination thereof. Flash drying or spray drying are particularly preferred. Spray-drying in particular provides a fine, uniform particle distribution, with very little particle content above 400 μm.

The drying step provides hornificated particles having a dryness level of 70% or more, and preferably 80% or more, more preferably 90% or more, most preferably 95% or more. The drying step suitably takes place for a time of 1-300 seconds, preferably 15-120 seconds, and more preferably 30-60 seconds.

The drying step may—depending on the drying method—take place at a temperature of 70-350° C., preferably 80-185° C., and more preferably 100-150° C. The composition comprising microfibrillated cellulose suitably has an initial dryness level of 60% or less, prior to the drying step.

The drying step preferably takes place in an inert atmosphere. Drying in an inert atmosphere reduces the formation of oxidised material with an off-white colour, thereby maintaining the colour of the hornificated particles closest to white. The MFC could also be dispersed in a co-solvent such as an alcohol solvent prior to drying.

The method may include a step of actively cooling the hornificated particles after the drying step. To prevent the particles from flocculating, the method may further comprise a step of adding an antiflocculating agent after said drying step.

As a final step, the method may include a step of formulating the hornificated particles into a personal care composition. Exfoliants are typically added into personal care products during the last formulation step under low shear. Re-dispersion of dry MFC particles was attempted using different conditions and it was verified that the particles do not re-disperse under low shear (see results, below).

All details of the particles described above are also relevant for the method of the invention, mutatis mutandis. In particular, the following aspects are preferred;
the method does not include a step of incorporating an antiflocculating agent prior to the drying step;
the method may provide hornificated particles having a water absorption capacity of less than 10 g water/g material; preferably less than 5 g water/g material; more preferably 1-5 g water/g material.
the method may provide hornificated particles having a porosity in the range 0-25%, preferably in the range 5-15%.

In a further embodiment, the use of hornificated particles comprising or consisting of microfibrillated cellulose (MFC), described herein as an abrasive material in a personal care composition is provided. Other uses include in a papermaking composition, a paint composition, pharmaceuticals or a food product. All details of the particles described above are also relevant for these uses, mutatis mutandis.

### Example 1

### Re-Dispersion Attempt of Dry MFC Particles

### Materials:

Four different types of MFC particles were obtained by drying using various technologies to a dryness level in the range 95-98%.

The MFC was obtained from Birch kraft pulp. The enzymatically pre-treated samples were refined and homogenized after enzymatic treatment and obtained at ca. 4 wt % solids content. The MFC that was not enzymatically pre-treated was only refined and obtained at a solids content ca. 4 wt %. Prior to drying the samples were dewatered to 20-30 wt % solids content by mechanical means.
MFC (enzymatically pre-treated) particles dried with single pass flash drying technology (SFD-MFC)
MFC particles dried with ring drying technology (RD-MFC)
MFC (enzymatically pre-treated) particles dried with spray drying technology (SD-MFC)
MFC (enzymatically pre-treated) particles oven dried and ball milled (OD-MFC)

### Experimental

The different dry MFC particles were added to beakers, followed by the addition of deionized water in order to obtain a 1 wt % solids content composition. The MFC particles and water mixtures were stirred under low shear (800 rpm) using an overhead mixer for 1, 5 and 10 min. After 1, 5 and 10 min of stirring, ca. 5 mL samples were taken for optical microscope observation. Low shear is particularly relevant, since in cosmetics formulations the exfoliating agents are added under low shear during the final formulation step.

One drop of each sample was added onto a microscope glass slide and covered with a cover glass. Optical microscope observations were conducted using a 5× magnification lens.

### Observations:

It was verified that the overall size and shape of the differently dried MFC particles was unaltered after 1, 5 and 10 min stirring under 800 rpm, except for the oven-dried particles, which, to some extent, started breaking into smaller particles. However, in all cases no visible signs of redispersion were noticed, what indicates that the particles are stable enough and suitable for application in cosmetic formulations.

FIG. 1 shows the optical microscopy images of SFD (single pass flash dried) MFC particles, RD (ring dried) MFC particles, SD (spray dried) MFC particles and OD (oven dried) MFC particles submitted to the redispersion tests at low shear conditions (800 rpm).

### Example 2

### Water Absorption of Dry MFC Particles

### Materials:

Four different types of MFC particles were obtained by drying using various technologies to a dryness level in the range 95-98%:
MFC (enzymatically pre-treated) particles dried with single pass flash drying technology (SFD-MFC)
MFC particles dried with ring drying technology (RD-MFC)
MFC (enzymatically pre-treated) particles dried with spray drying technology (SD-MFC)
MFC (enzymatically pre-treated) particles oven dried and ball milled (OD-MFC)

### Experimental

About 0.2 g of the different dry MFC particles were weighed on watch glasses. Deionized water was added dropwise until the materials could not absorb more water. Excess of water was carefully removed using blotting paper. The final weight of MFC particles plus water was measured and water absorption calculated according to the formula below:

Water absorption=(w_{MFCwet}−w_{MFCdry})/w_{MFCdry} (g water/g material)

In which w_{MFCdry} stands for the weight of the dry MFC particles and w_{MFCwet} stands for the final weight of MFC particles upon absorbing water.

### Observations:

It was verified that the different samples had low water absorption capacity in the range 1-5 g water/g material, which suggests extensive degree of hornification.

| | Water absorption |
|---|---|
| Dry MFC type | (g water/g) |
| SFD-MFC | 1.25 |
| RD-MFC | 2.45 |
| SD-MFC | 4.81 |
| OD-MFC | 2.35 |

(Benchmark: Water absorption of microcrystalline cellulose-based exfoliants: 1-4 g water/g.)

### Example 3

### Performance of MFC Particles as Exfoliants in Cosmetic Formulations

### Materials:

Four different types of MFC particles were obtained by drying using various technologies to a dryness level in the range 95-98%:
MFC (enzymatically pre-treated) particles dried with single pass flash drying technology (SFD-MFC)
MFC particles dried with ring drying technology (RD-MFC)
MFC (enzymatically pre-treated) particles dried with spray drying technology (SD-MFC)
MFC (enzymatically pre-treated) particles oven dried and ball milled (OD-MFC)

Commercial rinse-off product (basis product) and commercial scrub product containing cellulose acetate beads (benchmark).

### Experimental

The differently dried MFC particles were firstly manually fractionated through 400 and 750 μm sieves. The fractions <400 μm and 400-750 μm from the differently dried MFC particles (only <400 μm fraction in the case of the SD-MFC) were incorporated into the commercial rinse-off basis product using a spatula until a homogeneous dispersion was obtained. Seven different formulations with 1 wt % MFC particles were produced.

The formulations containing MFC and a benchmark commercial product containing cellulose acetate beads were transferred to 20 mL transparent plastic containers (labeled as formulation A to H).

| Formulation | Composition |
|---|---|
| Formulation A | Commercial basis product + |
| | 1% RD MFC particles < 400 μm |
| Formulation B | Commercial benchmark product (CA beads) + |
| | 9.5% commercial basis product (to control viscosity) |
| Formulation C | Commercial basis product + |
| | 1% OD MFC particles < 400 μm |
| Formulation D | Commercial basis product + |
| | 1% SFD MFC particles 400-750 μm |
| Formulation E | Commercial basis product + |
| | 1% SD MFC particles < 400 μm |
| Formulation F | Commercial basis product + |
| | 1% SFD MFC particles < 400 μm |
| Formulation G | Commercial basis product + |
| | 1% OD MFC particles 400-750 μm |
| Formulation H | Commercial basis product + |
| | 1% RD MFC particles 400-750 μm |

The performance of the exfoliant particles in the formulations were qualitatively tested by a panel group of 18 people (4 male and 14 female) with ages ranging 16-61 years old.

### Observations:

Overall, 74% of the people from the panel group preferred formulations containing MFC particles.

### Example 4

### Abrasiveness Level of Cosmetic Formulations Containing MFC Particles as Exfoliants

### Materials:

Same as in Example 3.

### Experimental

Same as in Example 3.

The same panel was asked to evaluate the different formulations according to the abrasiveness level: not abrasive, gentle, medium and hard.

### Observations:

FIG. 2 show the results from a panel evaluation of the abrasiveness level of formulations containing MFC particles.

In an assessment of abrasiveness, formulations G and H, which contained OD and RD MFC particles 400-750 μm, respectively, were perceived as having similar abrasiveness as the benchmark formulation B containing CA beads.

On the other hand, the formulation containing the SD MFC particles, which were the ones with lower particle size (<400 μm), was perceived as little or no abrasive, which could be good for facial scrubs.

Overall, the results indicate that hornificated MFC particles can be used as a replacement for commercial exfoliant in personal care compositions, with at least as good abrasiveness profile. Should lower or higher abrasiveness be required, MFC particles can be tailored accordingly.

Although the invention has been described with reference to a number of aspects, examples and embodiments, these aspects, examples and embodiments may be combined by the person skilled in the art, while remaining within the scope of the present invention.

1. Hornificated particles comprising:
at least 50 wt % of microfibrillated cellulose (MFC), wherein said hornificated particles comprise a dryness level of 61% or more and a water absorption capacity of less than 10 g water/g material.

2. The hornificated particles according to claim 1, wherein the dryness level comprises 70% or more.

3. The hornificated particles according to claim 1, wherein the hornificated particles comprising an average dry particle size (D90) of 1-2000 μm.

4. The hornificated particles according to claim 1, wherein the water absorption capacity comprises less than 5 g water/g material.

5. The hornificated particles according to claim 1, wherein the hornificated particles comprises a porosity in the range 0-25% 5-15%.

6. The hornificated particles according to claim 1, wherein a percentage of loose fibrils upon wetting is <5%.

7. The hornificated particles according to claim 1, additionally comprising natural fibres.

8. The hornificated particles according to claim 1, further comprising at least 70 wt % MFC.

9. The hornificated particles according to claim 1, wherein the hornificated particles do not comprise an antiflocculating agent.

10. The hornificated particles according to claim 1, wherein said microfibrillated cellulose (MFC) is native MFC.

11. A personal care composition comprising:
the hornificated particles according to claim 1.

12. The personal care composition according to claim 11, wherein the personal care composition is in the form of a liquid, and wherein the hornificated particles are dispersed in said liquid.

13. The personal care composition according to claim 11, wherein the personal care composition is in the form of a solid, wherein the hornificated particles are dispersed throughout said solid.

14. The personal care composition according to claim 11, further comprising particles which do not comprise MFC.

15. The personal care composition according to claim 11, wherein the comprises between 0.1-10 wt % of said hornificated particles.

16. The personal care composition according to claim 11, further comprising one or more surfactants.

17. The personal care composition according to claim 11, wherein the personal care composition is selected from a group consisting of: toothpastes, face scrubs, body scrubs, foot scrubs, and bath and shower products.

18. A method for producing hornificated particles comprising or consisting of microfibrillated cellulose (MFC), said method comprising the steps of:
drying a composition comprising at least 50 wt % of microfibrillated cellulose (MFC), so as to provide hornificated particles having a dryness level of 61% or more.

19. The method according to claim 18, wherein said hornificated particles having a dryness level of 70% or more.

20. The method according to any one of claim 18, wherein said drying step comprises spray drying, ring drying, flash drying, TurboRotor mill drying, oven drying, or a combination thereof.

21. The method according to claim 20, wherein the hornificated particles have a particle size of 1-2000 μm.

22. The method according to claim 18, wherein the composition comprising microfibrillated cellulose has an initial dryness level of 60% or less, prior to the drying step.

23. The method according to claim 18, wherein said drying step takes place in an inert atmosphere.

24. The method according to claim 18, further comprising a step of actively cooling the hornificated particles after the drying step.

25. The method according to claim 18, further comprising a step of adding an antiflocculating agent after said drying step.

26.-27. (canceled)
